(19) United States
(12) Patent Application Publication
(10) Pub. No.: US 2024/0320581 A1
(43) Pub. Date: Sep. 26, 2024

(54) SUPPLY AND LOGISTICS MODELLING SYSTEM FOR PANDEMIC RESPONSE

(71) Applicant: Logistics and Supply Chain MultiTech R&D Centre Limited, HK (HK)

(72) Inventors: Chi Hung TONG, HK (HK); Chin Chiu Chung, HK (HK); Kong SIT, HK (HK)

(21) Appl. No.: 18/187,298

(22) Filed: Mar. 21, 2023

(51) Int. Cl. G06Q 10/0631; G06Q 10/0833; G06Q 10/0835; G06Q 10/087

(52) U.S. Cl. CPC G06Q 10/06315; G06Q 10/0833; G06Q 10/08355; G06Q 10/087

(57) ABSTRACT

A supply and logistics modelling system for pandemic response including a computing apparatus comprising a processing unit and a memory unit, the processing unit arranged in communication with the memory unit, a user interface operatively coupled to the processing unit, the user interface configured to receive inputs from a user and present information to the user, the processing unit providing a supply and logistics operational model defining pandemic response actions, the user interface presenting one or more actions to guide a user in pandemic response actions, and wherein the model is an automated model.

### TECHNICAL FIELD

The present invention relates to a supply and logistics modelling system for pandemic response. The modelling system further allows a user to customise the model for pandemic response.

### BACKGROUND

Pandemics or epidemics can be scenarios that can stress supply chains. Pandemic and epidemic scenarios can have an acute impact on the drug supply chain i.e., the medication supply chain. Such large-scale disease scenarios can make it very difficult to manage the distribution of medications.

Various pandemic goods are often required to be supplied during pandemic scenarios e.g., medications, vaccines, food etc. Various software based logistics systems have been used by multiple operators. Information systems supporting counter pandemic actions i.e., supplying pandemic goods are required to be responsive, scalable, operable with multiple operators and provide data analytics.

Number of operating sites providing pandemic goods e.g., clinics or frontline operators providing mass vaccination programs each have used very specific and custom operation models. Very different workflows were devised to operate the respective programs. It takes excessive time to develop software systems to manage supply of pandemic goods. These individual systems do not often communicate with each other and managing the logistics of supply of pandemic goods becomes a challenge as there is no easy way to obtain an overall representation of the supply of pandemic goods. This can be especially challenging for authorities e.g., local, provincial, state or national governments or national health administrators to manage the supply of pandemic goods.

### SUMMARY OF THE INVENTION

The present invention relates to a supply and logistics modelling system and method that can be used to provide an improved model for managing pandemic response. Pandemic response relates to supply of pandemic goods e.g., medications or vaccines. The model defined provided by the system can be utilised by various operators or clinics to provide an improved way to manage distribution of medications or vaccines. The system also reduces the need to develop customised models for each user e.g., operator or clinic in order to manage distribution of pandemic goods such as, for example, vaccines or medications. Pandemic goods may also relate to other goods such as food, water, health supplements etc.

The modelling system further provides a fully customisable set of tools that allow a user to create customised models. The customised models can be created by the user based on selection or unselection of modules. This allows a user to customise the operational model for pandemic response or customise its components. The modules are a predefined set of modules. Each module corresponds to a component of the operational model. The customised models are a subset of an overall operational model that may be standardised for use by users. The standardised operational model reduces the need for users to develop their own models from the ground up thereby reducing costs, resource usage and improves speed. The model being standardised also improves tracking of the distribution of pandemic goods and improves logistics management. However, the model being customisable allows users the freedom to tailor the model to their situation.

In one aspect the present invention relates to a supply and logistics modelling system for pandemic response comprising:
a computing apparatus comprising a processing unit and a memory unit, the processing unit arranged in communication with the memory unit,
a user interface operatively coupled to the processing unit, the user interface configured to receive inputs from a user and present information to the user,
the processing unit providing a supply and logistics operational model defining pandemic response actions,
the user interface presenting one or more actions to guide a user in pandemic response actions, and wherein the model is an automated model.

In one example, the model or parts of the model are customisable by a user, the user interface configured to receive customisation inputs, and the processing unit configured to update the model or parts of the model according to the customisation inputs, wherein the custom model providing custom pandemic response actions.

In one example the pandemic response actions correspond to actions required to distribute pandemic goods to one or more operational sites and/or providing one or more public members at the one or more operational sites with the pandemic goods.

In one example the processing unit comprising a plurality of components defining the operation model, the components comprising:
an ordering interface presented on the user interface, the ordering interface configured to receive orders for pandemic goods from one or more users,
a dispatch controller configured to control dispatch and distribution of the pandemic goods,
a site operation module configured to track one or more operational tasks associated with one or more sites providing a pandemic goods,
an analytics engine configured to generate data related to the pandemic response actions.

The user interface is configured to present the data related to pandemic response actions.

The analytics engine is configured to generate data related to at least one of: the ordering of pandemic goods, or dispatch and distribution of pandemic goods, or operational tasks.

The supply and logistics modelling system for pandemic response further comprising:
a communications link operatively coupled to the processing unit,
the communications link configured to transmit data or one or other messages to a remote device or remote system.

In one example the ordering interface comprising:
a web ordering interface presented on the user interface to receive orders for pandemic goods from one or more users,
an inventory database storing current inventory levels of pandemic goods,
a resource planning module configured to assess orders and allocate pandemic goods to the one or more users based on the amount of inventory in the inventory database and the order amount.

The ordering interface may further optionally comprise: a program management module configured to assess the amount of pandemic goods available in the inventory, determine one or more order parameters and assign the amount of pandemic goods that can be provided to each user that has placed an order.

In one example the dispatch controller comprises:
a transport planning engine configured to calculate transportation routes to the users and payload details,
a transport interface configured to communicate, via the communications link, to the various transport providers the transportation routes and payload details.

In one example the site operation module defines a standardised process for each site for provision of pandemic goods, the site operation module further comprising a dashboard system for tracking pandemic good stock, issuance of pandemic goods and wastage.

The dashboard may be presented on the user interface and/or the dashboard may be presented on a remote user interface associated with the site operator.

In one example the system comprises: a central command interface configured to link to one or more governing entities, and the central command interface configured to transmit the data from the analytics engine to the governing entities.

In one example the system further comprising:
one or more data capture devices configured to generate tracking data related to provision and receipt of pandemic goods to the public members, wherein the data capture devices are hardware devices,
the data capture devices configured to communicate with the system via the communications link in the system,
the system receiving the tracking data, storing the tracking data and processing the tracking data.

Optionally the analytics engine is configured to process the tracking data and generate additional metrics.

In one example the supply and logistics modelling system for pandemic response further comprising:
a data repository storing the data from the analytics engine,
a data repository API interface configured to allow one or more remote parties to remotely access the data in the data repository to assess performance of the model in provision of pandemic goods.

Optionally the processing unit is configured to generate a customisation console and present the customisation console on the user interface and receive customisation inputs via the customisation console.

In one example the customisation console is configured to present a plurality of selectable predefined modules, each module corresponding to a component of the operational model, the modules being selectable or unselectable allowing a user to fully customise the operational model and its components, and the user configured to deploy the customised model for managing pandemic response actions.

In another aspect the present invention relates to a supply and logistics modelling method for pandemic response comprising the steps of:
providing a supply and logistics operational model defining pandemic response actions,
implementing the model, by a computing apparatus to control pandemic response actions,
presenting on a user interface one or more actions to guide a user in pandemic response actions wherein the model is an automated model.

In one example the model is executed automatically by a computing apparatus comprising a memory unit, processing unit, user interface and a communications link applying the model and the method comprising the steps of:
receiving orders for pandemic goods from one or more users, wherein the users are frontline operators or private clinics,
storing current inventory of pandemic goods in an inventory database
assessing received orders and allocating pandemic goods to one or more users based on the amount of inventory in the inventory database and the order amount,
calculating transportation routes to the users that ordered pandemic goods and payload details,
communicating to various transportation providers the transportation routes and payload details,
tracking the journey of payloads and confirming receipt of the ordered pandemic goods at each user,
tracking one or more operational tasks associated with one or more sites providing a pandemic goods, wherein the operational tasks are executed based in a standardised process for each site for provision of the ordered pandemic goods,
presenting data related to the operational tasks on a dashboard system,
generating data related to the pandemic response actions, wherein data comprises one or more of: the ordering of pandemic goods, or dispatch and distribution of pandemic goods, or operational tasks,
storing the data in the memory unit and/or storing the data in a remote memory unit,
presenting the data on the user interface and/or transmitting the data to one or more remote systems and/or providing an API to one or more remote systems to access the data.

Optionally the model is fully customisable and the method comprising the additional steps of:
presenting a plurality of selectable modules, each module corresponding to a component of the operational model, the modules being selectable or unselectable allowing a user to fully customise the operational model and its components,
updating the model based on the selections to generate a customised model,
deploying the customised model for managing pandemic response actions.

The term “pandemic” as used herein encompasses a pandemic or epidemic or any other disease that infects a large number of people.

### BRIEF DESCRIPTION OF THE DRAWINGS

Embodiments of the supply and logistics modelling system for pandemic response will now be described, by way of example, with reference to the accompanying drawings in which:

FIG. 1 illustrates an example method that is used for community testing programme and its operational workflow.

FIG. 2 illustrates an operational workflow for a vaccination programme.

FIG. 3 illustrates the different operational models in for different operators and the specific workflows for the different sites.

FIG. 4 illustrates a schematic diagram of an embodiment of a supply and logistics modelling system for pandemic response.

FIG. 5 illustrates a schematic diagram of the operational model for pandemic response.

FIG. 6 illustrates a flow chart for a supply and logistics modelling method for pandemic response.

FIG. 7 illustrates a flow chart for an example supply and logistics modelling method for pandemic response.

FIG. 8 illustrates an example of a customisation console to allow a user to create a custom operational model.

### DETAILED DESCRIPTION OF THE PREFERRED EMBODIMENTS

The present invention relates to a supply and logistics modelling system for pandemic response. The modelling system further allows a user to customise the model for pandemic response. In particular, the present invention relates to a supply and logistics modelling system for pandemic response that provides an improved model for managing supply of pandemic related goods.

Various software based logistics systems have been used by multiple operators. Information systems supporting counter pandemic actions i.e., supplying pandemic goods are required to be responsive, scalable, operable with multiple operators and provide data analytics.

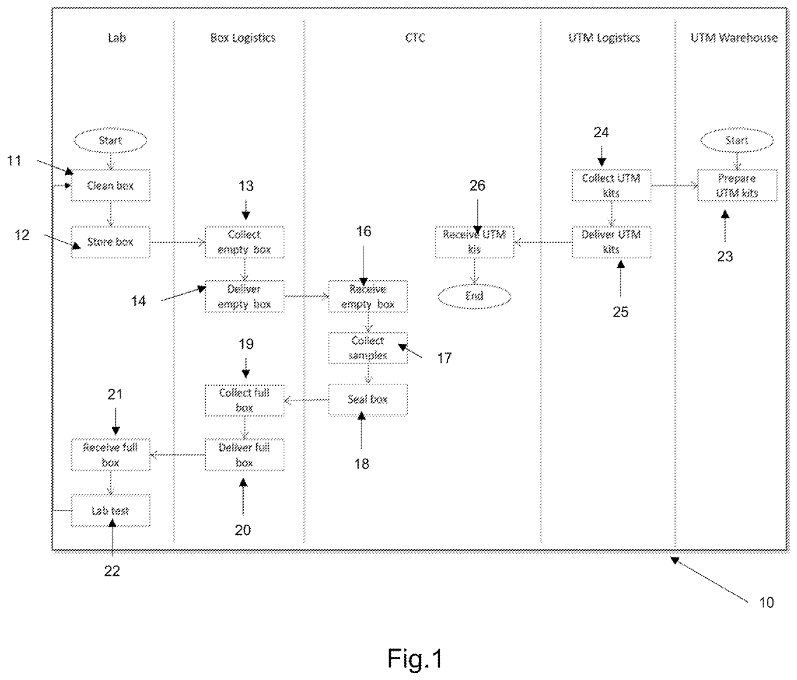

FIG. 1 illustrates an example method that is used for community testing programme. Referring to FIG. 1 the operational workflow illustrates various functions performed by the lab, box logistics provider, CTC (community testing centre), as well the testing kit provider (UTM). Referring to FIG. 1, the method for community testing 10. The method 10 comprises receiving a clean box at step 11. The box is stored at step 12. The box logistics provider performs collects the empty box at step 13. Step 14 comprises delivering the empty box. Step 16 comprises receiving y box. Step 17 comprises collecting samples. Step 18 comprises sealing the box. Steps 16 to 18 are performed by the CTC. Step 19 comprises collecting the full box. Step 20 comprises delivering the full box from the box logistics provider to the Lab. Step 21 comprises receiving the box and step 22 comprises performing the lab test. The method returns back to step 11. Steps 23 to 26 define the process of preparing UTM testing kits and delivering these to the CTC. Step 23 comprises the UTM warehouse preparing UTM test kits. Step 24 comprises collecting the UTM kit and step 25 comprises delivering UTM kits. Step 26 comprises the CTC receiving the UTM kits. This method is one example. Currently, there is no standardised method for community testing. There are currently multiple customised methods by each CTC.

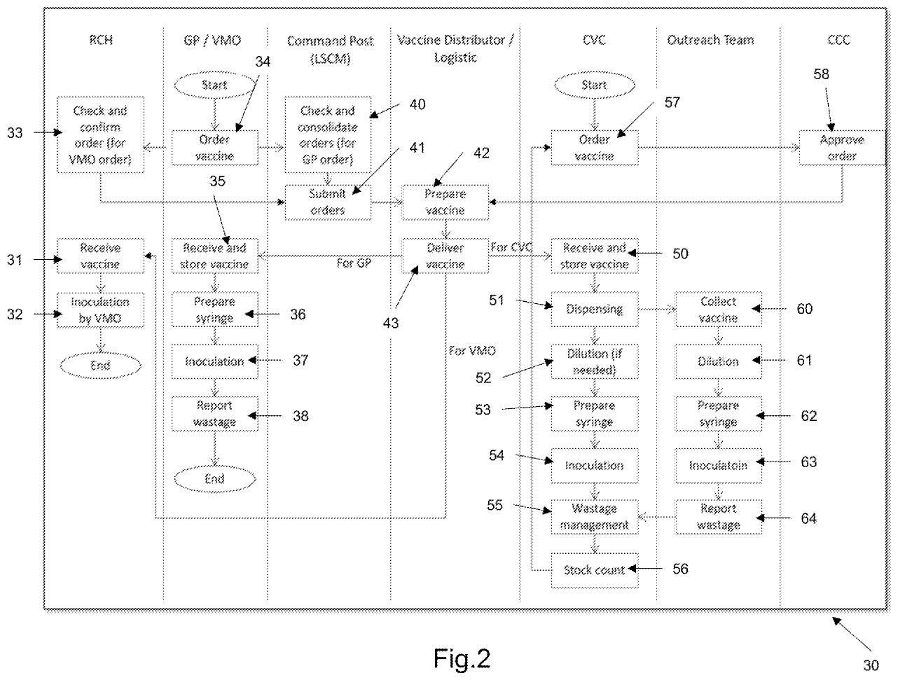

FIG. 2 illustrates an operational workflow for a vaccination programme 30. In this example the pandemic goods are vaccines (i.e., vaccine doses). Referring to FIG. 2 RCH stands for the residential care homes that inoculates patients that live in these care homes, e.g., senior citizens The method 30 as shown in FIG. 2 shows the steps performed by the RCH. Step 31 comprises receiving the vaccines from a vaccine distributor. Step 32 comprises inoculating by VMO. Step 33 comprises checking and confirming the order that is received.

The GP/VMO are private clinics that inoculate patients. Step 34 comprises ordering vaccines. The ordered vaccines can be provided to the RCH. Step 35 comprises receiving and storing the vaccines. Step 36 comprises preparing a syringe. Step 37 comprises inoculating patients. Step 38 comprises reporting wastage. Step 40 comprises checking and consolidating orders. Step 41 comprises submitting the orders. Steps 40 and 41 are preformed by the command post (LCSM). The command post (LSCM) is an organisation that consolidates orders for vaccinations and submits orders.

Step 42 comprises preparing vaccines. Step 43 comprises delivering vaccines. Steps 42 and 43 are performed by the vaccine distributor. CVCs are community vaccination centres. Steps 50 to 56 are performed by the CVC. Step 50 comprises receiving and storing vaccines from the CVC. Step 51 comprises dispensing vaccine doses. Step 52 comprises dilution if needed. Step 53 comprises preparing syringe. Step 54 comprises inoculating a patient. Step 55 comprises collecting waste and managing waste. Step 56 comprises counting remaining stock. Step 57 comprises ordering stock if stock levels are below a threshold. Steps 60 to 64 are performed by a community outreach team. Step 60 comprises collecting vaccines e.g., from a CVC or other provider. Step 61 comprise dilution of the vaccine. Step 62 comprises preparing a syringe. Step 63 comprises inoculating the patient. Step 64 comprises reporting wastage back to the CVC. Step 58 comprises approving an order by a CCC e.g., a central command or administrator.

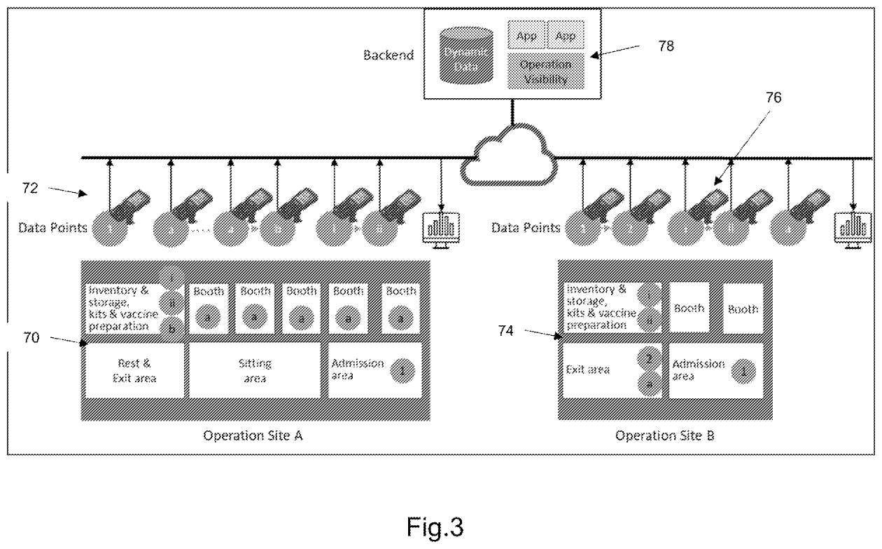

FIG. 3 illustrates the different operational models in for different operators and the specific workflows for the different sites. Very different workflows are devised to operate the respective programmes. There are multiple different operation models in different community centres run by different operators. Hence, data systems in each case are custom developed. Each operator has custom workflows and custom logistics management systems. Data is captured by hardware devices e.g., scanners. Operation site A has a completely different method of performing pandemic actions to operation site B. Site A has a method 70 comprising multiple elements. The data is captured by the data capture units 72. Site B has its own method 74. Method 74 is a unique operational method of operation site B. Data capture units 76 capture data from site B. The backend 78 comprises apps to process data and display data. The varying operational models require a lot of work in the backend systems that process this data. The data processing systems are not very flexible and resource intense due to being built to operate with many custom operating models. The variability can also lead to difficulty in assessing effectiveness of pandemic actions and makes it difficult to compare the performance of each operating site. Further each of the operating sites can be different in size and traffic. This lack of standardisation can make it very difficult, expensive and inefficient to perform pandemic actions e.g., vaccinating a population.

FIGS. 1 to 3 are prior art methods and illustrate the unstandardised and inefficient methods for pandemic actions e.g., vaccinations. Anti-pandemic actions i.e., pandemic response actions such as distributing pandemic goods demand the parties involved to be capable of supporting rapid development and deployment of the data and IT systems to different sites for operation in response to various pandemic conditions. This can take excessive time to develop such software systems and increase cost.

The present invention relates to a supply and logistics modelling system for pandemic response that is customisable for responsive deployment to various operators in providing pandemic goods. The modelling system provides an operational model that is substantially standardised thereby simplifying the logistics and management of pandemic goods. The model can be used by various community operation operators for logistics management in the provision of various programmes e.g., for vaccination programmes. The operational model is a standardised model that simplifies logistics management and make logistics management more efficient.

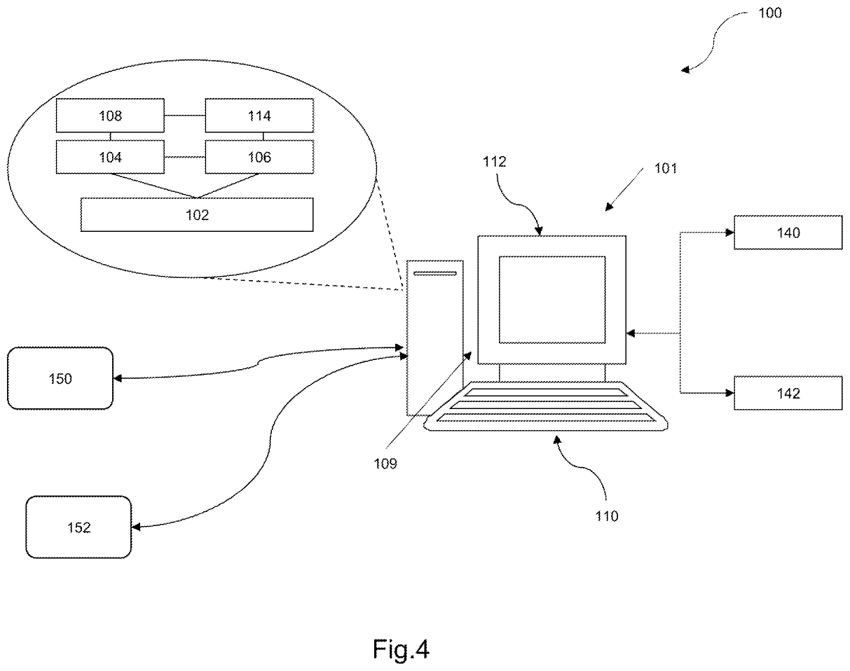

Referring to FIG. 4, an embodiment of the present invention is illustrated. This embodiment is arranged to provide a supply and logistics modelling system for pandemic response comprising: a computing apparatus comprising a processing unit and a memory unit, the processing unit arranged in communication with the memory unit, a user interface operatively coupled to the processing unit, the user interface configured to receive inputs from a user and present information to the user, the processing unit providing a supply and logistics operational model defining pandemic response actions, the user interface presenting one or more actions to guide a user in pandemic response actions, and wherein the model is an automated model. The automated model is useful since it can automatically track provision of pandemic goods e.g., vaccinations or medications.

The pandemic response actions correspond to actions required to distribute pandemic goods to one or more operational sites and/or providing one or more public members at the one or more operational sites with the pandemic goods. The user interface is configured to present the data related to pandemic response actions

The operational model or parts of the model are customisable by a user, the user interface configured to receive customisation inputs, and the processing unit configured to update the model or parts of the model according to the customisation inputs, wherein the custom model providing custom pandemic response actions.

In this example embodiment, the system may be implemented by a computing apparatus i.e., a computer or computing system having appropriate components such as a processing unit, memory unit and a user interface. The computer may be implemented by any computing architecture, including portable computers, tablet computers, stand-alone Personal Computers (PCs), smart devices, Internet of Things (IoT) devices, edge computing devices, client/server architecture, “dumb” terminal/mainframe architecture, cloud-computing based architecture, or any other appropriate architecture.

In another example the system may be implemented by multiple computing apparatuses or multiple computing devices. The multiple computing apparatuses may be arranged in a distributed architecture. The computing apparatus or apparatuses may be appropriately programmed to implement the invention.

In the illustrated example, the system is arranged to provide an improved operational model for managing supply and distribution of pandemic goods e.g., vaccines or medications. The system is arranged to provide an improved operational model defining pandemic response actions. The system is configured to present one or more actions to guide a user in pandemic response actions. The model is preferably an automated model that provides improved logistics management of pandemic goods for pandemic response. The automated model is useful since it can automatically track provision of pandemic goods e.g., vaccinations or medications.

FIG. 4 illustrates a schematic diagram of a supply and logistics modelling system 100 for pandemic response. FIG. 4 illustrates the system 100 comprising a computing apparatus 101 and may also comprise additional remote systems 140, 142. These computing systems are arranged in communication with the computing apparatus 101. The computing apparatus 101 may be configured to transmit data to the one or more systems 140, 142. Some examples of remote systems 140, 142 may be government systems or other organisations.

The computing apparatus 101 i.e., a computer system or computer server which is arranged to be implemented as an example embodiment of a supply and logistics modelling system 100 for pandemic response. In this embodiment the computing apparatus 101 includes suitable components necessary to receive, store and execute appropriate computer instructions. The components may include a processing unit 102, including Central Processing Unit (CPU), Math Co-Processing Unit (Math Processor), Graphic Processing Unit (GPUs) or Tensor processing unit (TPUs) for tensor or multi-dimensional array calculations or manipulation operations. The computing apparatus 101 comprises a memory unit. The computing apparatus 101 may comprise a read-only memory (ROM) 104, random access memory (RAM) 106, and input/output devices such as disk drives 108, input devices 110 such as an Ethernet port, a USB port, etc. The system comprises a user interface 109 configured to receive inputs from a user and present information to the user. The user interface may comprise input devices 110 may include a keyboard or keypads.

The system 100 may also comprise remote input devices e.g., one or more data capture devices 150, 152 configured to generate tracking data related to provision and receipt of pandemic goods to the public members, wherein the data capture devices are hardware devices. The data capture devices 150, 152 are configured to communicate with the system, more specifically the computing apparatus 101 via a communications link in the computing apparatus 101. The system is configured to receive the tracking data, store the tracking data and process the tracking data

The computing apparatus may comprise at least one display 112. The display 112 is part of the user interface. The display 112 may be a liquid crystal display, a light emitting display or any other suitable display. The display 112 may be a touchscreen that is configured to present data or information to a user. The touchscreen may also receive inputs from the user.

The computing apparatus 101 further comprises a communications links 114. The communications link 114 are configured to transmit data or one or other messages to a remote device or remote system

The computing apparatus 101 may include instructions that may be included in ROM 104, RAM 106 or disk drives 108 and may be executed by the processing unit 102. There may be provided a plurality of communication links 114 which may variously connect to one or more computing devices such as a server, personal computers, terminals, t wireless or handheld computing devices, Internet of Things (IoT) devices, smart devices, edge computing devices. At least one of a plurality of communications link may be connected to an external computing network through a telephone line or other type of communications link.

The computing apparatus 101 may include storage devices such as a disk drive 108 which may encompass solid state drives, hard disk drives, optical drives, magnetic tape drives or remote or cloud-based storage devices. The server 100 may use a single disk drive or multiple disk drives, or a remote storage service. The computing apparatus 101 may also have a suitable operating system which resides on the disk drive or in the ROM of the computing apparatus 101.

The computing apparatus may also provide the necessary computational capabilities to operate or to interface with a machine learning network, such as a neural networks, to provide various functions and outputs. The neural network may be implemented locally, or it may also be accessible or partially accessible via a server or cloud-based service. The machine learning network may also be untrained, partially trained or fully trained, and/or may also be retrained, adapted or updated over time. The operational model may be a neural network or another suitable machine learning model. The operational model defining pandemic response actions and the model defines a simplified and more efficient logistics management tool for supplying pandemic goods.

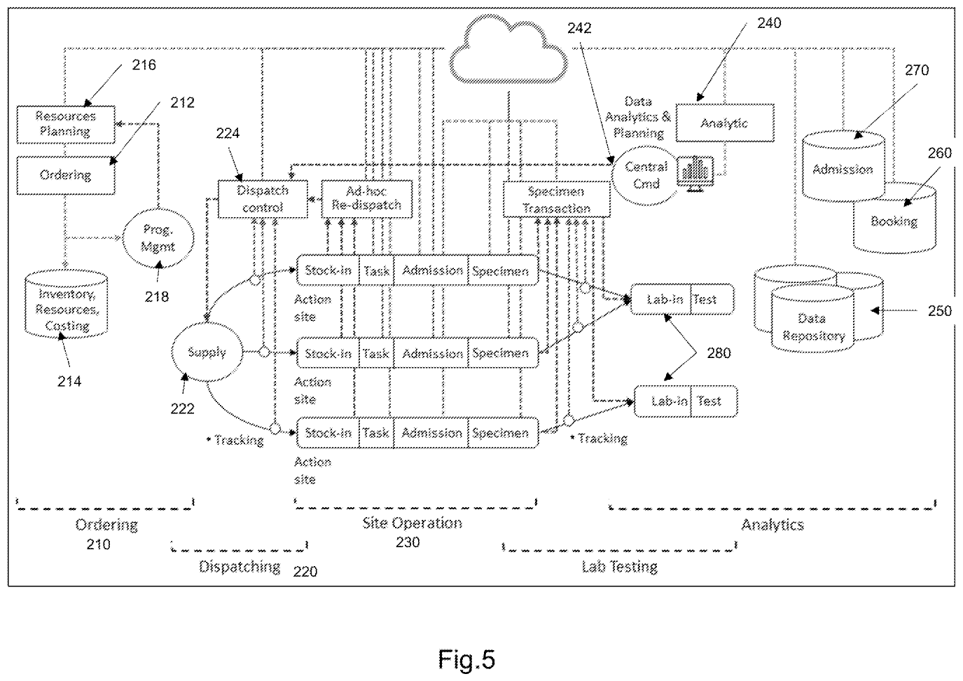

FIG. 5 illustrates a schematic diagram of the operational model 200 for pandemic response i.e., for managing logistics of pandemic goods during a pandemic event. The model may be implemented in a distributed computing apparatus or in a cloud system or in the computing apparatus 101 described earlier. The operational model 200 components are illustrated in FIG. 5. The operational model components may be implemented as software modules or software components. The software modules may be implemented as part of the computing apparatus 101. The operational model 200 may be stored in a memory unit and executed by the processing unit 102. The processing unit 102 may be programmed to generate and/or modify the operational model 200.

Alternatively, the operational model 200 and its components may be hardware modules that are part of the system 100. In a further alternative form, the operational model 200 may comprise a combination of hardware and software modules and the components may be part of the system 100. Optionally some or all the components the model 200 may be implemented by the computing apparatus 102.

Referring to FIG. 5, the operating model comprises an ordering interface 210. The ordering interface 210 is configured to receive orders for pandemic goods from one or more users. The users may be users of pandemic goods e.g., in the case of vaccines or medications the users may be GPS, clinics, community health centres, pharmacies etc.

The ordering interface 210 may be presented on a user interface associated with a device of the user. The ordering interface optionally be presented on the display 112. The ordering interface 210 may comprise a web ordering interface 212 that may be presented on the user interface or another suitable interface to receive orders for pandemic goods from one or more users.

In one example the ordering interface 210 may comprise web-based ordering systems e.g., a web interface for different frontline operators e.g., community vaccination centres. The ordering interface 210 may also comprise a web interface for orders and usage of private clinics or GPs etc.

The ordering interface may further comprise an inventory database 214 storing current inventory levels of pandemic goods. The database 214 may be stored in a memory unit or a remote memory unit e.g., a cloud system.

The ordering interface 210 may further comprise a resource planning module 216 configured to assess orders and allocate pandemic goods to the one or more users based on the amount of inventory in the inventory database and the order amount.

The ordering interface 210 further comprises a program management module 218 configured to assess the amount of pandemic goods available in the inventory. The program management module 218 is configured to determine one or more order parameters and assign the amount of pandemic goods that can be provided to each user that has placed an order. Optionally the ordering interface 210 may comprise a logistics and inventory interface for a service provider

The operational model 200 comprises a dispatch controller 220 that is configured to control dispatch and distribution of the pandemic goods. The dispatch controller 220 comprises a transport planning engine 222 configured to calculate transportation routes to the users and payload details. The transport planning engine 222 is configured to provide a trucking plan for a service provider e.g., a trucking company. The dispatch controller 220 further comprises a transport interface 224 configured to communicate, via the communications link, to the various transport providers the transportation routes and payload details. FIG. 5 illustrates the transport interface 224 as a dispatch control module.

The operational model 200 comprises a site operation module 230 configured to track one or more operational tasks associated with one or more sites providing a pandemic goods.

The model 200 may also define a lab testing logistics system 280. The lab testing system 280 may define a standardised approach for collection and delivery of tests to laboratories.

The site operation module 230 defines a standardised process for each site for provision of pandemic goods, the site operation module further comprising a dashboard system for tracking pandemic good stock, issuance of pandemic goods and wastage. As shown in FIG. 5 each site i.e., each operator such as a GP practices, CVC, private clinics are provided with a standardised process through the site operation module. FIG. 5 shows 3 sites with the same process. Each process has stock inwardly received followed by one or more tasks, followed by admission of patients, collecting specimens and providing the specimens to labs. The specimens could be tracked as indicated in FIG. 5.

The operational model 200 comprises an analytics engine 240 configured to generate data related to the pandemic response actions. The analytics engine 240 is configured to generate data related to at least one of: the ordering of pandemic goods, or dispatch and distribution of pandemic goods, or operational tasks

The model 200 comprises a central command interface 242 configured to link to one or more governing entities. The central command interface 242 is configured to transmit the data from the analytics engine 240 to the governing entities. The operational model 200 further comprises a plurality of data repositories. In the illustrated example the model 200 comprises a data repository 250. The data repository 250 is configured to store the data from the analytics engine 240. A data repository API may be provided as part of the system. The data repository API interface configured to allow one or more remote parties to remotely access the data in the data repository to assess performance of the model in provision of pandemic goods.

The system 100 may comprise one or more data capture devices configured to generate tracking data related to provision and receipt of pandemic goods to the public members, wherein the data capture devices are hardware devices. The data capture devices may be configured to communicate with the system via the communications link 114 in the system 100. The system 100 is configured to receive the tracking data, store the tracking data and process the tracking data. Optionally, the analytics engine is configured to process the tracking data and generate additional metrics.

The operational model 200 may comprise a booking repository 260 or database. The booking repository 260 may be configured to store bookings e.g., bookings of pandemic goods. The model 200 may optionally comprise an admissions database 27. The admissions database 270 may store admissions information related to various patients.

The model or parts of the model are customisable by a user. The user interface may be configured to receive customisation inputs, and the processing unit may be configured to update the model or parts of the model according to the customisation inputs. The custom model provides custom pandemic response actions. The user interface is configured to present the data related to pandemic response actions.

The system 100, in particular, the processing unit 102 may be configured to generate a customisation console. The customisation console may be presented on the user interface and receive customisation inputs via the customisation console. The customisation console is configured to present a plurality of selectable modules. Each module corresponds to a component of the operational model. The modules may be selectable or unselectable allowing a user to fully customise the operational model and its components. The user may selectively deploy the customised model for managing pandemic response actions.

The operational model 200 shown in FIG. 5 operates in an automated manner. A user can provide various inputs associated with pandemic goods e.g., quantity of vaccines, the ordered amounts, locations that have ordered vaccines etc. The model can also use additional pandemic related parameters such for example, infection rate as increase/decrease, total population, eligible population for vaccines, number of vaccinated etc. The model can use inputs and parameters to provide guidance on pandemic actions. The model may apply machine learning techniques to optimise the model and structure of the model. The model is configured to automatically guide a user in pandemic response actions. The model may also be automatically generated based on the inputs and parameters from a user.

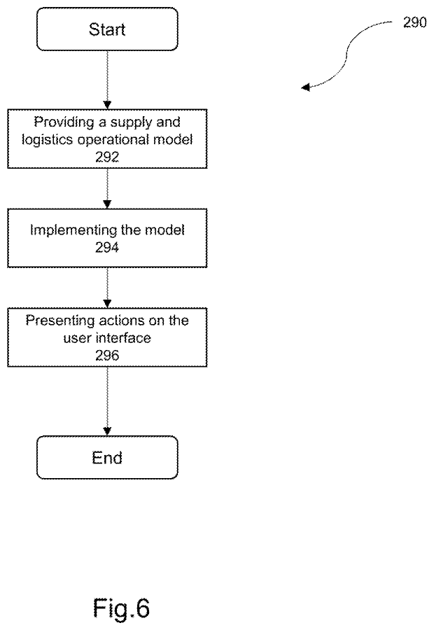

The system 100 is further configured to implement a supply and logistics modelling method for pandemic response. FIG. 6 illustrates a flow chart of the method 290. The method 290 is implemented by the components of the system 100. The method 290 can be implemented by the various components of the system 100. The supply and logistics modelling method 290 comprises the steps of: providing a supply and logistics operational model defining pandemic response actions at step 292. The operational model may be as per the model illustrated in FIG. 5. Step 294 comprises implementing the model, by a computing apparatus 101 to control pandemic response actions. Step 296 comprises presenting on a user interface one or more actions to guide a user in pandemic response actions. The method is an automated method that automatically generates and implements the model. The user can initiate the method and it will automatically initiate the model and apply the model to various input parameters.

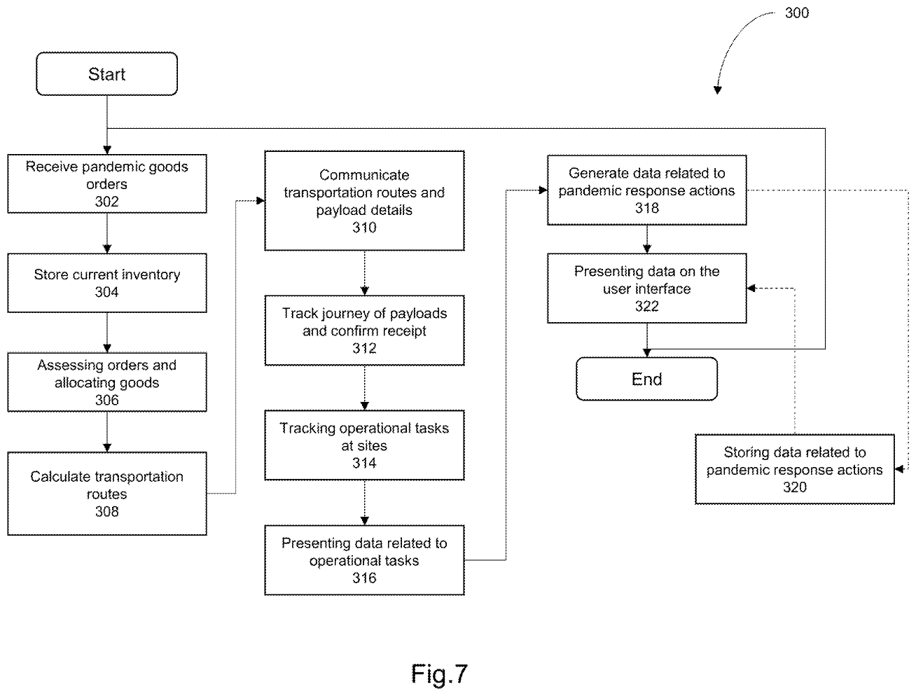

FIG. 7 illustrates an example supply and logistics modelling method 300 for pandemic response. The method 300 is executed automatically by the system. In particular, the method steps may be implemented by the computing apparatus comprising a memory unit, processing unit, user interface and a communications link. The method 300 is also implemented by the components of the operational model.

The method 300 can commence at step 302. Step 302 comprises receiving orders for pandemic goods from one or more users, wherein the users are frontline operators or private clinics. Orders may be received through the user interface, via the ordering interface 210 presented to the user. Step 304 comprises storing current inventory of pandemic goods in an inventory database. The inventory database may be part of the ordering interface.

Step 306 comprises assessing received orders and allocating pandemic goods to one or more users based on the amount of inventory in the inventory database and the order amount. Step 306 may be executed by the resource planning module 216. Steps 308 and 310 may be executed by the dispatch controller 220. Step 308 comprises calculating transportation routes to the users that ordered pandemic goods and payload details. Step 308 is performed by the transport planning engine 222. Step 310 comprises communicating to various transportation providers the transportation routes and payload details. The transportation routes may be communicated via the transport interface 224.

Step 312 comprises tracking the journey of payloads and confirming receipt of the ordered pandemic goods at each user. The payload is the amount of pandemic goods being transported. Step 314 comprises tracking one or more operational tasks associated with one or more sites providing a pandemic goods, wherein the operational tasks are executed based in a standardised process for each site for provision of the ordered pandemic goods. Step 314 may be controlled by the site operation module 230. The tracking of site operation tasks may be automated. Further the module 230 may also provide instructions regarding site operation tasks to users. These instructions may relate to steps and tasks to be performed and the order these should be performed in. The module 230 presents a standardised and more efficient process.

Step 316 comprises presenting data related to the operational tasks on a dashboard system. Step 318 comprises generating data related to the pandemic response actions, wherein data comprises one or more of: the ordering of pandemic goods, or dispatch and distribution of pandemic goods, or operational tasks. The data may be generated by the analytics engine 240. Optionally the method comprises step 320. Optional step 320 comprises storing the data in the memory unit and/or storing the data in a remote memory unit. Step 322 comprises presenting the data on the user interface and/or transmitting the data to one or more remote systems and/or providing an API to one or more remote systems to access the data.

A supply and logistics modelling method for pandemic response may be used to customise the operational model. The model is fully customisable, and the method comprises the steps of: presenting a plurality of selectable modules, each module corresponding to a component of the operational model, the modules being selectable or unselectable allowing a user to fully customise the operational model and its components, updating the model based on the selections to generate a customised model, and deploying the customised model for managing pandemic response actions.

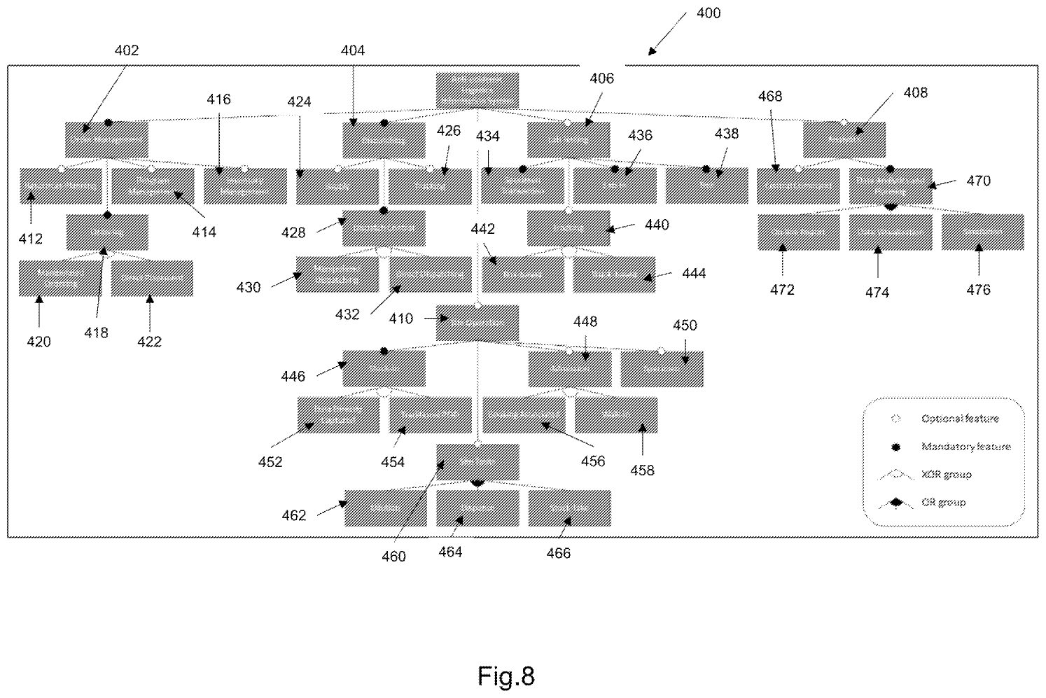

FIG. 8 illustrates an example of a customisation console 400. The customisation console 400 presents on a screen. The console 400 presents multiple selectable modules. The modules can be selected by a user in order to create the operational module. The modules selected form a standardised operational model. Each module corresponds to a component of the operational model. The modules may be selectable or unselectable allowing a user to fully customise the operational model and its components. The user may selectively deploy the customised model for managing pandemic response actions.

Referring to FIG. 8, the high-level modules correspond to order management 402, dispatching 404, lab testing 406, analytics 408 and a site operation module 410. Each high-level module has a number of sub modules. These sub modules can be selectable. Some modules are mandatory features for the operational model, and these are indicated with black dots as per the legend. There are modules that are optional which are indicated by white circles. The console 400 illustrates a decision tree of modules. The tree structure defines the relationships between various modules. Several modules may be related in an exclusive OR i.e., XOR or an inclusive OR. This is illustrated by the colour coded branches.

Referring to FIG. 8, under the order management module 402 may correspond to the ordering interface 210 described earlier. The order management module 402 may comprise two optional sub modules for resource planning 412, program management 414 and inventory management 416. These three are optional modules a user can select. A mandatory module for order management is the ordering module 418. Under the ordering module there are two modules that are selectable. These are the manipulated ordering module 420 and the direct procurement module 422.

The dispatching module 404 corresponds to the dispatch controller 220 described earlier. Under the dispatching module 404 there is available a supply module 424 or a tracking module 426 that are selectable. These are both optional. A mandatory sub module is the dispatch control module 428. This may correspond to the transport planning engine from earlier. The names are different, but the function of the module is the same. The dispatch control module 428 (i.e., the transport planning engine), may comprise two selectable sub modules titled manipulated dispatching 430 or direct dispatching 432. These modules may further provide dispatching and dispatch control functions.

The lab testing module 406 may correspond to the lab testing system 280. Three mandatory sub modules comprise specimen transaction module 434, the Lab-in module 436 and the testing module 438. Each module may define specific functions that can be selected by the user as part of the operational model. Module 406 may optionally include the tracking module 440. This may further comprise two additional optional modules for tracking which are box-based tracking 442 or truck based tracking 444.

The site operation module 410 corresponds to site operation module 230 described earlier. The mandatory Stock-in module 446 is required. This tracks the stock in coming. The stock in module may comprise two additional optional sub modules data directly captured 452 or traditional POD 454. Optional sub modules include admissions module 448 and a specimen module 450. The admission module may comprise the optional booking association module 456 and walk in module 458. The booking module 456 tracks bookings and the walk-in module 458 tracks walk ins. The site operation module 410 may include the sub module site tasks 460 that can define various tasks. The tasks may be customisable. Further optional sub modules from the site tasks module are dilution module 462, dispense module 464 and the stock take module 466. The dilution module and dispense module may define software functions for diluting specimens and dispensing goods e.g., vaccines.

The analytics module may correspond to the analytics engine 240. The analytics module 408 may comprise the optional module central command module 468 that allows a central organisation to access data e.g., through an API. The central organisation may be a government or health authority or another suitable administrator. The analytics module comprises the compulsory i.e., mandatory module data analytics and planning 470. This module defines various analytics functions and planning functions. The module 470 may comprise three optional modules: on-line reporting module 472, data visualisation module 474 and simulation module 476.

The console 400 shown in FIG. 8 illustrates a decision tree structure that can be followed when building a custom operational model. The console view allows a user to systematically manage variability and commonality among information systems supporting various pandemic actions. The console and customisability allow synthesis of system variants from the set of reusable software modules. This enables rapid deployment to support pandemic actions and allows adequate customisation to account for the various needs of a user. The mandatory fields ensure standardisation of the overall model similar to the generalised operational model 200 of FIG. 5. A common set of core modules are always implemented thereby the model more efficient that current practice of creating a new model for each pandemic action.

The system 100 can be configured and the model may be constructed by selection of features or modules respecting the constraints of each user. The system consists of a set of assets e.g., source code files, test case, documentation etc.

Although not required, the embodiments described with reference to the Figures can be implemented as application programming interface (API) or as a series of libraries for use by a developer or can be included within another software application, such as a terminal or personal computer operating system or a portable computing device operating system. Generally, as program modules include routines, programs, objects, components and data files assisting in the performance of particular functions, the skilled person will understand that the functionality of the software application may be distributed across a number of routines, objects or components to achieve the same functionality desired herein.

It will also be appreciated that where the methods and systems of the present invention are either wholly implemented by computing system or partly implemented by computing systems then any appropriate computing system architecture may be utilised. This will include stand alone computers, network computers and dedicated hardware devices. Where the terms “computing system” and “computing device” are used, these terms are intended to cover any appropriate arrangement of computer hardware capable of implementing the function described.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

The phrases ‘computer-readable medium’ or ‘machine-readable medium’ as used in this specification and claims should be taken to include, unless the context suggests otherwise, a single medium or multiple media. Examples of multiple media include a centralised or distributed database and/or associated caches. These multiple media store the one or more sets of computer executable instructions. The phrases ‘computer-readable medium’ or ‘machine-readable medium’ should also be taken to include any medium that is capable of storing, encoding or carrying a set of instructions for execution by a processor of a computing device and that cause the processor to perform any one or more of the methods described herein.

The computer-readable medium is also capable of storing, encoding or carrying data structures used by or associated with these sets of instructions. The phrases ‘computer-readable medium’ and ‘machine readable medium’ include, but are not limited to, portable to fixed storage devices, solid-state memories, optical media or optical storage devices, magnetic media, and/or various other mediums capable of storing, containing or carrying instruction (s) and/or data. The ‘computer-readable medium’ or ‘machine-readable medium’ may be non-transitory.

Also, it is noted that the embodiments may be described as a process that is depicted as a flowchart, a flow diagram, a structure diagram, or a block diagram. Although a flowchart may describe the operations as a sequential process, many of the operations can be performed in parallel or concurrently. In addition, the order of the operations may be rearranged. A process is terminated when its operations are completed. A process may correspond to a method, a function, a procedure, a subroutine, a subprogram, etc., in a computer program. When a process corresponds to a function, its termination corresponds to a return of the function to the calling function or a main function.

Furthermore, embodiments may be implemented by hardware, software, firmware, middleware, microcode, or any combination thereof. When implemented in software, firmware, middleware or microcode, the program code or code segments to perform the necessary tasks may be stored in a machine-readable medium such as a storage medium or other storage (s). A processor may perform the necessary tasks. A code segment may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

One or more of the components and functions illustrated the figures may be rearranged and/or combined into a single component or embodied in several components without departing from the scope of the invention. Additional elements or components may also be added without departing from the scope of the invention. Additionally, the features described herein may be implemented in software, hardware, as a business method, and/or combination thereof.

In its various aspects, embodiments of the invention can be embodied in a computer-implemented process, a machine (such as an electronic device, or a general purpose computer or other device that provides a platform on which computer programs can be executed), processes performed by these machines, or an article of manufacture.

1. A supply and logistics modelling system for pandemic response comprising:
a computing apparatus comprising a processing unit and a memory unit, the processing unit arranged in communication with the memory unit,
a user interface operatively coupled to the processing unit, the user interface configured to receive inputs from a user and present information to the user,
the processing unit providing supply and logistics operational model defining pandemic response actions,
the user interface presenting one or more actions to guide a user in pandemic response actions, and wherein the model is an automated model.

2. A supply and logistics modelling system for pandemic response in accordance with claim 1, wherein the model or parts of the model are customisable by a user, the user interface configured to receive customisation inputs, and the processing unit configured to update the model or parts of the model according to the customisation inputs, wherein the custom model providing custom pandemic response actions.

3. A supply and logistics modelling system in accordance with claim 1, wherein the pandemic response actions correspond to actions required to distribute pandemic goods to one or more operational sites and/or providing one or more public members at the one or more operational sites with the pandemic goods.

4. A supply and logistics modelling system in accordance with claim 3, wherein the system comprising a plurality of components defining the operation model, the components comprising:
an ordering interface presented on the user interface, the ordering interface configured to receive orders for pandemic goods from one or more users,
a dispatch controller configured to control dispatch and distribution of the pandemic goods,
a site operation module configured to track one or more operational tasks associated with one or more sites providing a pandemic goods,
an analytics engine configured to generate data related to the pandemic response actions.

5. A supply and logistics modelling system in accordance with claim 4, wherein the user interface is configured to present the data related to pandemic response actions.

6. A supply and logistics modelling system in accordance with claim 4, wherein the analytics engine is configured to generate data related to at least one of: the ordering of pandemic goods, or dispatch and distribution of pandemic goods, or operational tasks.

7. A supply and logistics modelling system for pandemic response in accordance with claim 1, further comprising:
a communications link operatively coupled to the processing unit,
the communications link configured to transmit data or one or other messages to a remote device or remote system.

8. A supply and logistics modelling system for pandemic response in accordance with claim 5, wherein the ordering interface comprising:
a web ordering interface presented on the user interface to receive orders for pandemic goods from one or more users,
an inventory database storing current inventory levels of pandemic goods,
a resource planning module configured to assess orders and allocate pandemic goods to the one or more users based on the amount of inventory in the inventory database and the order amount.

9. A supply and logistics modelling system for pandemic response in accordance with claim 8, wherein the ordering interface further comprising: a program management module configured to assess the amount of pandemic goods available in the inventory, determine one or more order parameters and assign the amount of pandemic goods that can be provided to each user that has placed an order.

10. A supply and logistics modelling system for pandemic response in accordance with claim 4, wherein the dispatch controller comprises:
a transport planning engine configured to calculate transportation routes to the users and payload details,
a transport interface configured to communicate, via the communications link, to the various transport providers the transportation routes and payload details.

11. A supply and logistics modelling system for pandemic response in accordance with claim 4, wherein the site operation module defines a standardised process for each site for provision of pandemic goods, the site operation module further comprising a dashboard system for tracking pandemic good stock, issuance of pandemic goods and wastage.

12. A supply and logistics modelling system for pandemic response in accordance with claim 11, wherein the dashboard is presented on the user interface and/or the dashboard is presented on a remote user interface associated with the site operator.

13. A supply and logistics modelling system for pandemic response in accordance with claim 4, wherein the system comprises: a central command interface configured to link to one or more governing entities, and the central command interface configured to transmit the data from the analytics engine to the governing entities.

14. A supply and logistics modelling system for pandemic response in accordance with claim 1, wherein the system further comprising:
one or more data capture devices configured to generate tracking data related to provision and receipt of pandemic goods to the public members, wherein the data capture devices are hardware devices,
the data capture devices configured to communicate with the system via the communications link in the system,
the system receiving the tracking data, storing the tracking data and processing the tracking data.

15. A supply and logistics modelling system for pandemic response in accordance with claim 14, wherein the analytics engine is configured to process the tracking data and generate additional metrics.

16. A supply and logistics modelling system for pandemic response in accordance with claim 4, wherein the system further comprising:
a data repository storing the data from the analytics engine,
a data repository API interface configured to allow one or more remote parties to remotely access the data in the data repository to assess performance of the model in provision of pandemic goods.

17. A supply and logistics modelling system for pandemic response in accordance with claim 2, wherein
the processing unit is configured to generate a customisation console, and,
present the customisation console on the user interface and receive customisation inputs via the customisation console.

18. A supply and logistics modelling system for pandemic response in accordance with claim 17, wherein the customisation console is configured to present a plurality of selectable predefined modules, each module corresponding to a component of the operational model, the modules being selectable or unselectable allowing a user to fully customise the operational model and its components, and the user configured to deploy the customised model for managing pandemic response actions.

19. A supply and logistics modelling method for pandemic response comprising the steps of:
providing a supply and logistics operational model defining pandemic response actions,
implementing the model, by a computing apparatus to control pandemic response actions,
presenting on a user interface one or more actions to guide a user in pandemic response actions wherein the model is an automated model.

20. A supply and logistics modelling method for pandemic response in accordance with claim 19, wherein the model is executed automatically by a computing apparatus comprising a memory unit, processing unit, user interface and a communications link applying the model comprising the steps of:
receiving orders for pandemic goods from one or more users, wherein the users are frontline operators or private clinics,
storing current inventory of pandemic goods in an inventory database
assessing received orders and allocating pandemic goods to one or more users based on the amount of inventory in the inventory database and the order amount,
calculating transportation routes to the users that ordered pandemic goods and payload details,
communicating to various transportation providers the transportation routes and payload details,
tracking the journey of payloads and confirming receipt of the ordered pandemic goods at each user,
tracking one or more operational tasks associated with one or more sites providing a pandemic goods, wherein the operational tasks are executed based in a standardised process for each site for provision of the ordered pandemic goods,
presenting data related to the operational tasks on a dashboard system,
generating data related to the pandemic response actions, wherein data comprises one or more of: the ordering of pandemic goods, or dispatch and distribution of pandemic goods, or operational tasks,
storing the data in the memory unit and/or storing the data in a remote memory unit,
presenting the data on the user interface and/or transmitting the data to one or more remote systems and/or providing an API to one or more remote systems to access the data.
